(19) United States
(12) Patent Application Publication
(10) Pub. No.: US 2025/0283874 A1
(43) Pub. Date: Sep. 11, 2025

(54) METHODS OF DETERMINING A HEALTH STATUS OF A DOG BASED ON ONE OR MORE BIOMARKERS, AND METHODS OF TREATING A MORTALITY RISK IDENTIFIED BY THE HEALTH STATUS

(71) Applicant: SOCIETE DES PRODUITS NESTLE S.A., Vevey (CH)

(72) Inventors: SEBASTIEN HERZIG, Morges (CH); PHILIPP GUT, Geneve (CH); LORANE TEXARI, Morges (CH); PASCAL STEINER, St. Louis, MO (US); YUANLONG PAN, Chesterfield, MO (US)

(21) Appl. No.: 18/859,295

(22) PCT Filed: Apr. 27, 2023
(86) PCT No.: PCT/EP2023/061058

§ 371 Date: Oct. 23, 2024

Related U.S. Application Data
(60) Provisional application No. 63/363,754, filed on Apr. 28, 2022.
(60) Provisional application No. 63/408,560, filed on Sep. 21, 2022.

(51) Int. Cl. G01N 33/50; G01N 33/569; G01N 33/573; G01N 33/66; G01N 33/68; G01N 33/72; G01N 33/84

(52) U.S. Cl. CPC G01N 33/5094; G01N 33/56972; G01N 33/573; G01N 33/66; G01N 33/6827; G01N 33/721; G01N 33/84; G01N 2333/765; G01N 2333/9123; G01N 2333/916

(57) ABSTRACT

The present invention relates to a method for determining a mortality risk and/or probability of a healthy lifespan of a dog; said method comprising determining the level of one or more biomarker(s) in one or more samples obtained from the dog, wherein the one or more biomarker(s) is selected from white blood cell count, serum albumin, serum alkaline phosphatase, serum creatine kinase, haemoglobin, haematocrit, mean corpuscular haemoglobin, serum glucose, mean red cell volume, serum globulin, serum calcium, platelet count, and/or red blood cell count.

### FIELD OF THE INVENTION

The present invention relates to a method for determining the health status, in particular the mortality risk and/or probability of a longer healthy lifespan, of a dog based on biomarkers.

### BACKGROUND TO THE INVENTION

The ability to determine information regarding the health of a dog, is desirable to inform about the dog's general health and well-being. For example, the ability to determine information about the dog's health may be helpful in assessing whether the dog would benefit from a dietary or supplement-based intervention.

Chronological age is known to be a major indicator of general health status, with increasing chronological age associated with reduced health. However, depending on genetics, nutrition, and lifestyles, individuals may age slower or faster than their chronological age. Chronological age may therefore not always reflect an individual's rate of aging or risk of reduced health. On the other hand, the biological age of an individual (based on e.g. clinical biochemistry and cell biology measures) can vary compared to others of the same chronological age. Methods for determining biological age may be helpful for identifying individuals at risk of age-related disorders earlier than would be expected based on their chronological age (see e.g. WO2019/165064).

However, there is a need for further methods of determining information regarding the health of dogs.

### SUMMARY OF THE INVENTION

The present invention relates to a method for quantifying the health status of a dog based on biomarkers, in particular blood biomarkers. The method enables a determination of mortality risk and/or probability of a healthy lifespan for a dog through assessment of the dog's phenotypic age.

Existing methods that use blood biomarkers to assess the health status of dogs determine biological age based on correlations between blood biomarkers and chronological age. Calculating the biological age of an animal may comprise determining differences in the level of one or more biomarker(s) compared to the expected level of the biomarker(s) at a given chronological age. Such methods are therefore based on the use of chronological age as the primary indicator of overall health.

In contrast, the present invention takes into account the direct predictive value of the described biomarkers on mortality risk and/or probability of a healthy lifespan. By way of example, a given biomarker may not directly correlate with chronological age, but may be indicative of a particular pathological condition and thus an increased mortality risk and/or a probability of a reduced healthy lifespan. The present methods may thus be described as identifying the phenotypic age (PhenoAge) of a dog. As such, the biomarkers of the present invention do not necessarily correlate with chronological age, but are related to the difference between phenotypic and chronological age of the dog.

The present invention provides a method for determining a mortality risk and/or probability of a healthy lifespan of a dog; said method comprising determining the level of one or more biomarker(s) in one or more samples obtained from the dog.

In one aspect the present invention provides a method for determining a mortality risk and/or probability of a healthy lifespan of a dog; said method comprising determining the level of one or more biomarker(s) in one or more samples obtained from the dog, wherein the one or more biomarker(s) is selected from white blood cell count, serum albumin, serum alkaline phosphatase, serum creatine kinase, haemoglobin, haematocrit, mean corpuscular haemoglobin, serum glucose, mean red cell volume, serum globulin, serum calcium, platelet count, and/or red blood cell count.

The present inventors have shown that each of these biomarkers is individually correlated with phenotypic age and is thus predictive of mortality risk and/or probability of a healthy lifespan.

Determining a mortality risk may refer to determining a likelihood that a dog will live for a longer or shorter period of time compared to an equivalent dog of—for example—the same chronological age, sex and breed. Accordingly, the present methods may determine the probability of a health span or a healthy lifespan for a dog compared to an equivalent dog of—for example—the same chronological age, sex and breed. Suitably, mortality risk may be equated to the probability of a healthy lifespan for the dog; wherein a decreased mortality risk is equated to an increased probably of longer healthy lifespan for the dog or an increased mortality risk is equated to a decreased probability of longer healthy lifespan for the dog. The mortality risk may be represented as the difference between phenoage and chronological age (phenoage advance) of the dog. For example, an increase in phenoage compared to chronological age may be indicative of an increased mortality risk for the dog; whilst a decrease in phenoage compared to chronological age may be indicative of a decreased mortality risk for the dog.

In one embodiment, the method may comprise determining the white blood cell count in one or more samples obtained from the dog. The method may further comprise determining the level of one or more biomarkers selected from serum albumin, serum alkaline phosphatase, serum creatine kinase, haemoglobin, haematocrit, mean corpuscular haemoglobin, serum glucose, mean red cell volume, serum globulin, serum calcium, platelet count, and/or red blood cell count in one or more samples.

In one embodiment, the method may comprise determining the serum albumin level in one or more samples obtained from the dog. The method may further comprise determining the level of one or more biomarkers selected from white blood cell count, serum alkaline phosphatase, serum creatine kinase, haemoglobin, haematocrit, mean corpuscular haemoglobin, serum glucose, mean red cell volume, serum globulin, serum calcium, platelet count, and/or red blood cell count in one or more samples.

In one embodiment, the method may comprise determining the serum alkaline phosphatase level in one or more samples obtained from the dog. The method may further comprise determining the level of one or more biomarkers selected from white blood cell count, serum albumin, serum creatine kinase, haemoglobin, haematocrit, mean corpuscular haemoglobin, serum glucose, mean red cell volume, serum globulin, serum calcium, platelet count, and/or red blood cell count in one or more samples.

In one embodiment, the method may comprise determining the serum creatine kinase level in one or more samples obtained from the dog. The method may further comprise determining the level of one or more biomarkers selected from white blood cell, serum albumin, serum alkaline phosphatase, haemoglobin, haematocrit, mean corpuscular haemoglobin, serum glucose, mean red cell volume, serum globulin, serum calcium, platelet count, and/or red blood cell count in one or more samples.

In one embodiment, the method may comprise determining the haemoglobin level in one or more samples obtained from the dog. The method may further comprise determining the level of one or more biomarkers selected from white blood cell, serum albumin, serum alkaline phosphatase, serum creatine kinase, haematocrit, mean corpuscular haemoglobin, serum glucose, mean red cell volume, serum globulin, serum calcium, platelet count, and/or red blood cell count in one or more samples.

In one embodiment, the method may comprise determining the haematocrit level in one or more samples obtained from the dog. The method may further comprise determining the level of one or more biomarkers selected from white blood cell, serum albumin, serum alkaline phosphatase, serum creatine kinase, haemoglobin, mean corpuscular haemoglobin, serum glucose, mean red cell volume, serum globulin, serum calcium, platelet count, and/or red blood cell count in one or more samples.

In one embodiment, the method may comprise determining the mean corpuscular haemoglobin level in one or more samples obtained from the dog. The method may further comprise determining the level of one or more biomarkers selected from white blood cell, serum albumin, serum alkaline phosphatase, serum creatine kinase, haemoglobin, haematocrit, serum glucose, mean red cell volume, serum globulin, serum calcium, platelet count, and/or red blood cell count in one or more samples.

In one embodiment, the method may comprise determining the serum glucose level in one or more samples obtained from the dog. The method may further comprise determining the level of one or more biomarkers selected from white blood cell, serum albumin, serum alkaline phosphatase, serum creatine kinase, haemoglobin, haematocrit, mean corpuscular haemoglobin, mean red cell volume, serum globulin, serum calcium, platelet count, and/or red blood cell count in one or more samples.

In one embodiment, the method may comprise determining the mean red cell volume in one or more samples obtained from the dog. The method may further comprise determining the level of one or more biomarkers selected from white blood cell, serum albumin, serum alkaline phosphatase, serum creatine kinase, haemoglobin, haematocrit, mean corpuscular haemoglobin, serum glucose, serum globulin, serum calcium, platelet count, and/or red blood cell count in one or more samples.

In one embodiment, the method may comprise determining the serum globulin level in one or more samples obtained from the dog. The method may further comprise determining the level of one or more biomarkers selected from white blood cell, serum albumin, serum alkaline phosphatase, serum creatine kinase, haemoglobin, haematocrit, mean corpuscular haemoglobin, serum glucose, mean red cell volume, serum calcium, platelet count, and/or red blood cell count in one or more samples.

In one embodiment, the method may comprise determining the serum calcium level in one or more samples obtained from the dog. The method may further comprise determining the level of one or more biomarkers selected from white blood cell, serum albumin, serum alkaline phosphatase, serum creatine kinase, haemoglobin, haematocrit, mean corpuscular haemoglobin, serum glucose, mean red cell volume, serum globulin, platelet count, and/or red blood cell count in one or more samples.

In one embodiment, the method may comprise determining the platelet count in one or more samples obtained from the dog. The method may further comprise determining the level of one or more biomarkers selected from white blood cell, serum albumin, serum alkaline phosphatase, serum creatine kinase, haemoglobin, haematocrit, mean corpuscular haemoglobin, serum glucose, mean red cell volume, serum globulin, serum calcium, and/or red blood cell count in one or more samples.

In one embodiment, the method may comprise determining the red blood cell count in one or more samples obtained from the dog. The method may further comprise determining the level of one or more biomarkers selected from white blood cell, serum albumin, serum alkaline phosphatase, serum creatine kinase, haemoglobin, haematocrit, mean corpuscular haemoglobin, serum glucose, mean red cell volume, serum globulin, serum calcium, and/or platelet count in one or more samples.

In one embodiment, the one or more biomarkers may comprise white blood cell count, serum albumin and serum alkaline phosphatase. Advantageously, this combination of three biomarkers has been determined to provide a notable prediction of mortality risk and/or probability of a healthy lifespan. The predictive ability may be further increased by incorporating one or more of the additional biomarkers selected from serum creatine kinase, haemoglobin, haematocrit, mean corpuscular haemoglobin, serum glucose, mean red cell volume, serum globulin, serum calcium, platelet count, and red blood cell count.

In one embodiment, the one or more samples is a blood sample, e.g. a blood plasma sample.

In one embodiment, the present invention provides a method for determining a mortality risk and/or probability of a healthy lifespan of a dog; said method comprising:
a. determining the level of the following biomarkers; white blood cell count, serum albumin, serum alkaline phosphatase, serum creatine kinase, haemoglobin, haematocrit, mean corpuscular haemoglobin, serum glucose, mean red cell volume, and serum globulin in one or more samples obtained from the dog; and
b. determining a phenotypic age (Phenoage) of the dog using formula (1):

$$Phenoage = ln ( \frac{{γ}_{breed} * {e}^{xb} * \{ {e}^{γ * age} - 1 \}}{{e}^{\{ breed * {β}_{breed 2} \} + \{ sex * {β}_{sex 2} \} + {β}_{02}} * γ} + 1 ) * \frac{1}{{γ}_{breed}}$$

where xb is the sum of the value of each biomarker(s), sex and breed multiplied by their respective coefficients according to formula (2):

$$xb = {\sum }_{u = 1}^{p} {x}_{u} {β}_{u} + {β}_{0}$$

wherein sex is coded as a numerical value with 0 for female and 1 for male,
wherein breed is coded as a numerical value with 0 for small breeds and 1 for medium breeds,
and wherein the phenotypic age is used to determine a mortality risk and/or probability of a healthy lifespan for the dog.

Suitably, determining that the phenoage of the dog is greater than its chronological age is indicative of a higher mortality risk. Suitably, determining that the phenoage of the dog is less than its chronological age is indicative of a reduced mortality risk. Suitably, determining that the phenoage of the dog is greater than its chronological age is indicative of a reduced probability of a longer healthy lifespan. Suitably, determining that the phenoage of the dog is less than its chronological age is indicative of an increased probability of a longer healthy lifespan.

The invention further relates to a method for selecting a lifestyle or dietary regime for a dog, the method comprising:
a. performing the method as described in the first aspect of the invention; and
b. selecting a suitable lifestyle or dietary regime based on the mortality risk and/or probability of a healthy lifespan determined in step a.

The invention further provides a method for determining the efficacy of a lifestyle or dietary regime for reducing the mortality risk and/or increasing the probability of a healthy lifespan of a dog, said method comprising: a. performing the method as described in the first aspect of the invention; b. applying a change in lifestyle or dietary regime to the dog; c. performing the method as described in the first aspect of the invention after a time period of applying the change in lifestyle or dietary regime; and d. determining if there has been a change in mortality risk and/or probability of a healthy lifespan for the dog between step a. and step c.

The invention further provides a dietary intervention for use in reducing the mortality risk and/or increasing the probability of a healthy lifespan of a dog, wherein the dietary intervention is administered to a dog with a mortality risk determined by the method of the present invention.

The invention also provides the use of a dietary intervention to reduce the determined mortality risk and/or increase the probability of a healthy lifespan of a dog, wherein the dietary intervention is administered to a dog with a mortality risk and/or probability of a healthy lifespan determined by the method of the present invention.

The invention further provides a computer-readable medium comprising instructions that when executed cause one or more processors to perform the method of the present invention.

The invention also provides a computer system for determining a mortality risk and/or probability of a healthy lifespan of a dog, the computer system programmed to perform one or more of the steps of: determine the mortality risk and/or probability of a healthy lifespan of a dog given the level of one or more biomarker(s) in a sample from the dog, wherein the one or more biomarker(s) is selected from white blood cells count, serum albumin, serum alkaline phosphatase, serum creatine kinase, haemoglobin, haematocrit, mean corpuscular haemoglobin, serum glucose, mean red cell volume; serum globulin, serum calcium, platelet count, and/or red blood cell count; and generating a report of the mortality risk and/or probability of a healthy lifespan of the dog.

The invention further provides a computer program product comprising computer implementable instructions for causing a programmable computer to determine the mortality risk and/or probability of a healthy lifespan of a dog given the level of one or more biomarker(s) from the dog, wherein the one or more biomarker(s) is selected from white blood cells count, serum albumin, serum alkaline phosphatase, serum creatine kinase, haemoglobin, haematocrit, mean corpuscular haemoglobin, serum glucose, mean red cell volume; serum globulin, serum calcium, platelet count, and/or red blood cell count.

Advantageously, embodiments of the present invention in which levels of multiple biomarkers are determined enable the determination of mortality risk and/or probability of a healthy lifespan based on markers of multiple organ systems and functions. Accordingly, the use of multiple biomarkers advantageously encompassed a range of potential organ dysfunctions.

Evaluating the mortality risk or probability of survival for a dog allows one to test several aspects of the animal's wellbeing. First, it can predict whether this animal is more likely to need a dietary or supplement-based intervention. Second, it can be used to test the efficacy of a dietary or supplement-based intervention on aging. It also provides a specific set of biomarkers that can be tested to evaluate the mortality risk or survival probability of an animal and that are routinely tested in vet clinic or offices.

### DESCRIPTION OF DRAWINGS

FIG. 1—Identification of blood biomarkers predictive of mortality risk. A cox proportional hazard model was fit for each of the 28 biomarkers assessed, including sex and breed class (small or medium). Values are adjusted for the p. value of each parameter to account for multiple comparison (by false discovery rate (fdr)). Parameters show are those with an adjusted fdr below 0.05.

FIG. 2—Demonstration of biomarkers that contribute to the predictive ability of the multi-parameter model for determining phenoage.

FIG. 3—Difference between phenoage and chronological age (phenoage advance) is associated with a significant increase in mortality risk.

FIG. 4—Difference in survival of dogs stratified by high or low median PhenoAge advance.

FIG. 5—Phenoage advance (delta with chronological age) changes mid-life with a calorie-restricted diet. This changes earlier in females than males.

FIG. 6—Stratification of under 7 and over 7 year old dogs reveals a significant difference in phenoage advance in older compared to younger dogs.

FIG. 7—Dogs fed two calorie-restricted diets for 6 months showed a statistically significant reduction in PhenoAge.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

The methods and systems disclosed herein can be used by veterinarians, health-care professionals, lab technicians, pet care providers and so on.

### Subject

The present methods are directed to canine subjects. Accordingly, the subject of the present invention is a dog.

Suitably, the dog may have a body condition score (BCS) of at least 7 (for example using the American Animal Hospital Association (AAHA)—9 point scale; AAHA Guidelines; Nutritional Assessment Body and Muscle Condition Score).

### Breed

The present methods may utilise information regarding the breed of the dog. The dog may be categorised as a toy, small, medium, large or giant breed—for example. Suitably, the dog breed may be categorised based on the weight of the dog. Suitably, the dog breed may be categorised based on the average weight of a dog for a given breed.

The dogs may be categorized based on genetic information obtained for the dog by DNA sequencing or SNP detection. For example, the dog may be categorized based on the clade of breeds to which it belongs.

Additionally or alternatively, the dog may be of mixed breed. The mixed breed dog may be categorized based on the weight of the dog. For example, the mixed breed dog can be classified as small, medium, large, or giant.

Additionally or alternatively, the mixed breed dog may be categorized based on genetic information such as DNA sequence, SNP, haplotypes or haplogroups. For example, the mixed breed dog can be categorized based on the closest breed or clade by genetic analysis.

Additionally or alternatively, genetic information such as DNA sequences, SNP, haplotypes or haploblocks that are known to be associated with breed identity can be used to categorise mixed breed or pure breed dogs.

Additionally or alternatively, genetic information such as DNA sequences, SNP, haplotypes or haploblocks that are known to be associated longevity can be used to categorise mixed breed or pure breed dogs.

Additionally or alternatively, the mixed-breed or pure breed dog can be categorized as robust or athletic based on morphometric measurements (for example, as disclosed in U.S. Pat. No. 8,091,509 to Perez-Camargo et al. and entitled “Method for improving dog food” or US2017/0042194 to Bouthegourd et al. and entitled “Methods using morphometric measurements of a small dog to improve food for the small dog,” both herein incorporated by reference in their entireties).

Preferably, the dog may be categorised as a small or medium breed. Preferably, the categorisation is determined by the average weight of adult dogs of this breed. Preferably, a breed with an average weight below 10 kg is categorised as a small breed and/or a breed with an average weight above 10 kg is categorised as a medium breed.

### Sex

Suitably, the sex of the dog may be classified as male or female.

### Chronological Age

Chronological age may be defined as the amount of time that has passed from the subject's birth to the given date. Chronological age may be expressed in terms of years, months, days, etc.

Suitably, the present method may be applied to a dog of any chronological age. In certain embodiments, the dog may be at least about 2 years old. Suitably, the dog may be at least about 2, at least about 3, at least about 4, at least about 5, at least about 6, at least about 7, at least about 8, at least about 9 or at least about 10 years old.

Suitably, the dog may be at least about 7 years old.

### Sample

The present invention comprises a step of determining the level of one or more biomarkers in one or more samples obtained from a subject.

Preferably, the sample is derived from blood. The sample may contain a blood fraction or may be whole blood. The sample preferably comprises blood plasma and/or serum. Techniques for collecting samples from a subject are well known in the art.

A suitable sample may be selected based on the biomarker(s) to be determined. By way of example, if the biomarkers are to be determined using a complete blood count (cbc), for example as part of a standard clinical complete blood count (cbc), a whole blood sample should be used.

The present method may be performed on one or more samples obtained from the subject. For example, the method may be performed using a first sample obtained at a given time point and a second sample obtained following a time interval after the first sample was obtained. The method may be performed more than once, on samples obtained from the same dog over a time period. For example, samples may be obtained repeatedly once per month, once a year, or once every two years. Suitably, the samples may be obtained around once per year (e.g. during an annual veterinary health check). This may be useful in determining the effects of a particular treatment or change in lifestyle—such as a dietary intervention or a change in exercise regime.

In one embodiment, the level of one or more biomarkers may be determined prior to a change in lifestyle (e.g. a dietary product intervention or a change in exercise regime). In another embodiment, the level of one or more biomarkers may be determined prior to, and after the e.g. dietary product intervention or change in exercise regime. The biomarker level may also be determined at predetermined times throughout the e.g. dietary product intervention or change in exercise regime. These predetermined times may be periodic throughout the e.g. dietary product intervention or change in exercise regime, e.g. every day or three days, or may depend on the subject being tested.

### Determining the Level of One or More Biomarkers in the Sample

The biomarkers used in the present invention can be determined using standard methods in the art and are typically measured as part of standard blood tests to determine the disease status of an animal. For example, the biomarkers are commonly determined as part of a standard clinical complete blood count (cbc) and standard clinical blood chemistry analysis.

A complete blood count provides information about blood cells and their properties; for example red blood cells, white blood cells, and platelets. An example complete blood count can comprise an automated process using flow cytometry or Coulter counter to determine cell number in the blood. In addition to determining cell count, such automated systems may also be capable of determining other blood biomarker readings depending on their complexity. Such systems are able to simultaneously measure blood cell counts as well as red blood cell volume, hemoglobin level, mean corpuscular hemoglobin level and hematocrit. For example, IDEXX Laboratories provide a hematology analyzer capable of determining white blood cell count (WBC), red blood cell count (RBC), platelet count (PLT), hemoglobin (HGB), hematocrit (HCT), mean red cell volume (MCV) and mean corpuscular hemoglobin (MCH) (IDEXX Laboratories Inc., ProCyte Dx Hematology Analyzer).

The levels of other biomarkers unrelated to blood cells, such as serum protein/enzyme and/or molecule biomarker levels, can be measured using chemical tests, in particular using automated chemistry analyser systems. These methods may utilize colorimetry-based approaches for quantification. For example, IDEXX Laboratories provide an automated chemistry analyzer able to quantify serum Albumin, serum Alkaline Phosphatase, serum Creatine Kinase, serum glucose, serum globulin, and serum Calcium (IDEXX Laboratories Inc., Catalyst One Chemistry Analyzer).

Accordingly, methods for determining the level of a biomarker used in the present invention may comprise assays that result in spectrophotometric changes (for example, chemical or antibody-linked changes that result in detectable signals at certain wavelengths). Such tests can be highly automated and efficient, and form the basis of many normal veterinary health check.

Suitably, the biomarker level may be determined after overnight fasting and measured using standard veterinary clinical practice.

The level of the individual biomarker species in the sample may be measured or determined by any suitable method known in the art. For example, mass spectrometry (MS), antibody detection methods, e.g. enzyme-linked immunoabsorbent assay (ELISA), non-antibody protein scaffolds (e.g. fibronectin scaffolds), radioimmuno-assay (RIA), or aptamers may be used. Other spectroscopic methods, chromatographic methods, labelling techniques, or quantitative chemical methods may also be used.

Suitable antibodies for use in methods described above are known in the art and/or may be generated using known techniques. Suitable test methods for detecting antibody levels include, but are not limited to, an immunoassay such as an enzyme-linked immunosorbent assay, radioimmunoassay, Western blotting and immunoprecipitation.

### Biomarkers

### White Blood Cell Count

White blood cells, also termed leukocytes, are a type of cell that are found in the blood. They have various immune-related functions, dependent on their sub-type: monocytes, lymphocytes, neutrophils, basophils and eosinophils. White blood cells contain a nucleus, and have a variable cell-shape that is also dependent on sub-type. White blood cell count is the number of this type of cell per volume of blood.

Methods of measuring white blood cell count, typically expressed in thousands of cells per microliter (10\hat{ }3/uL), are known in the art. White blood cell count measurements can be done manually on a blood smear using staining and microscopy techniques, but can also be carried out as part of an automated complete blood count (CBC). IDEXX Laboratories provide an automated hematology analyzer capable of white blood cell count measurements.

Suitably, increased white blood cell count may be associated with a negative effect on reducing mortality risk. Accordingly, increased white blood cell count may be associated an increased mortality risk.

### Serum Albumin

Serum Albumin is a globular protein found in the blood. It is a 65 kDa protein comprised of three homologous domains. Albumin regulates oncotic pressure, preventing loss of fluid from the blood to the tissues, and acting as a transport protein for fatty acids, bilirubin, heme, heavy metals, hormones and certain drugs. Albumin is highly abundant in the blood, accounting for 25-50% of total plasma protein by weight, and is produced by the liver. Abnormally high or low levels of albumin in the blood can be indicative of liver or kidney disease.

Methods of measuring serum albumin levels, typically expressed in grams per decilitre (g/dL), are well known in the art, and include zone electrophoresis, dye-binding assays involving bromocresol green (BCG) or bromocresol purple (BCP), and ELISA methods. For example, Eagle Biosciences offer an ELISA-based assay for canine serum albumin (Eagle Biosciences Inc., product code SKU: CAE49-K01). Stokol et al. describe the use of automated systems using a BCG-binding assay in determining serum Albumin levels in dogs in a clinical setting (Vet Clin Pathol. 2001; 30(4):170-176). Additionally, IDEXX Laboratories offer an automated chemistry analyzer that can conduct measurement of serum Albumin levels as part of a combined blood test.

Suitably, increased serum albumin levels may be associated with a positive effect on reducing mortality risk. Accordingly, increased serum albumin levels may be associated a reduced mortality risk.

### Serum Alkaline Phosphatase

Alkaline Phosphatase (ALP) is an enzyme that has an important role in liver metabolism and in skeletal development. It is a 86 kDa homodimeric protein. High levels of this protein in the blood can be indicative of liver damage or bone disease.

Methods of measuring serum alkaline phosphatase levels, typically expressed as units per litre (U/L), are well known in the art, and most often consist of quantification of enzymatic activity via colorimetric assay. An automated chemistry analyser is available from IDEXX Laboratories that is capable of measuring serum alkaline phosphatase levels.

Suitably, increased serum alkaline phosphatase levels may be associated with a negative effect on reducing mortality risk. Accordingly, increased serum alkaline phosphatase levels may be associated an increased mortality risk.

### Serum Creatine Kinase

Creatine Kinase (CK) is an enzyme that is predominantly found in muscles. This enzyme converts creatine and ATP into phosphocreatine for use in rapid energy generation during muscular contraction. The presence of high levels of this enzyme in the blood can be indicative of muscle damage.

Methods of determining creatine kinase levels in the blood, typically expressed in international units per litre (IU/L), are well known in the art, and examples of which use enzymatic assays to quantify creatine kinase levels. The automated chemistry analyzer provided by IDEXX Laboratories is an example of a commercially available tool to measure serum creatine kinase levels.

Suitably, increased creatine kinase levels may be associated with a negative effect on reducing mortality risk. Accordingly, increased creatine kinase levels may be associated an increased mortality risk.

### Haemoglobin

Hemoglobin is a transport protein in red blood cells. It consists of a tetramer of two alpha chains and two beta chains. Each peptide chain binds a heme group, which consists of a porphyrin ring with an iron ion bound. This group can reversibly bind oxygen which allows hemoglobin to function as an oxygen-transport carrier protein.

Methods of determining hemoglobin levels, typically expressed in grams per decilitre (g/dL), are well known in the art. The International Committee for Standardization in Haematology describe a standardized method utilising spectrophotometric determination of hemoglobin cyanide (Br J Haematol. 1967 April; 13:71-5) and this approach can be used in commercially available automated chemical analyzers such as that provided by IDEXX Laboratories.

Suitably, increased haemoglobin levels may be associated with a positive effect on reducing mortality risk. Accordingly, increased haemoglobin may be associated a reduced mortality risk.

### Haematocrit

Hematocrit is the percentage by volume of red blood cells in the blood. Hematocrit levels that fall outside normal values can be indicative of diseases or conditions that result in a greater or lesser proportion of red blood cells in the blood than normal. A high hematocrit can be indicative of conditions such as dehydration, for example; whilst a low hematocrit can be indicative of anemia, hemolysis or decreased production of red blood cells.

Methods of determining haematocrit, typically expressed as a percentage of blood volume (%), are well known in the art. Measurement can be carried out manually using packed cell volume by centrifuging blood in a microhematocrit tube. Alternatively, haematocrit can be calculated from mean red cell volume and the red blood cell count, both of which can be measured directly by modern hematology analyzers in a standard complete blood count (CBC).

Suitably, increased haematocrit levels may be associated with a positive effect on reducing mortality risk. Accordingly, increased haematocrit may be associated a reduced mortality risk.

### Mean Corpuscular Haemoglobin

Mean corpuscular hemoglobin (MCH) is the average mass of haemoglobin per red blood cell. MCH values that are outside normal ranges can be indicative of certain diseases such as macrocytic or hypochromic anemias.

Methods of measuring MCH, typically expressed in picograms (pg), are well known in the art, and commonly comprise calculation from observed values of hemoglobin level and red blood cell count that can be measured during a complete blood count (CBC) carried out using a hematology analyzer as described above.

Suitably, increased MCH may be associated with a positive effect on reducing mortality risk. Accordingly, increased MCH may be associated a reduced mortality risk.

### Serum Glucose

Serum glucose is a measure of the amount of glucose present in the blood. The level of glucose in the blood is controlled by hormones such as insulin to keep glucose levels within normal ranges. When glucose levels in the blood are outside normal levels it can be indicative of disease such as diabetes mellitus.

Methods of measuring serum glucose levels, typically expressed in milligrams per decilitre mg/dL, are well known in the art. Most glucose assays are photometric, and there are many available commercial devices. For example, Acon Pharmaceuticals offer a veterinary glucose monitoring system (Acon Phramaceuticals Inc., CentriVet® GK), and Carradini et al. describe the use of continuous glucose monitoring devices in dogs (Abbott Laboratories, FreeStyle Libre).

Suitably, increased serum glucose levels may be associated with a negative effect on reducing mortality risk. Accordingly, increased serum glucose levels may be associated an increased mortality risk.

### Mean Red Cell Volume

Mean red cell volume, or mean corpuscular volume (MCV), is a measure of the average volume of a red blood cell in the blood. MCV is a diagnostic criteria that can categorize a possible anemia into micro-, normo-, or macrocytic anemia and may help to identify an underlying disease or disorder. High MCV can be indicative of disorders such as vitamin B12 deficiency, whereas low MCV can be indicative of disorders such as iron deficiency.

Methods of determining mean red cell volume, typically expressed in femtoliters (fL), are well known in the art. It can be calculated from the other measured values of hemocrit and red blood cell count, though modern hematology analyzers (e.g. IDEXX Laboratories Inc., ProCyte Dx Hematology Analyzer) are able to directly measure the MCV as part of a complete blood count (CBC).

Suitably, increased MCV may be associated with a positive effect on reducing mortality risk. Accordingly, increased MCV may be associated a reduced mortality risk.

### Serum Globulin

Serum globulin is a measure of the concentration of globular protein in the blood. Globular proteins are secreted mainly by the liver and a smaller proportion are secreted by immune cells. Albumin is the most abundant of the serum globulins. The remaining serum globulins can be separated into fractions based on their behaviour in electrophoresis separation methods. Immunoglobulins are an important part of the immune system and are secreted by immune cells. Examples of other serum globulins are immune system proteins such as complement, hormones and carrier proteins such as ferritin. Changes in the total level of serum globulin proteins can be indicative of certain conditions or diseases. An overall rise in serum globulins can indicate infection and an inflammatory immune response, whereas a fall in levels can be indicative of bleeding, gastrointestinal disease or severe malnutrition.

Methods of determining serum globulin levels, typically expressed in grams per decilitre (g/dL), are well known in the art. For example, chemical and physical methods are described in Tothova et al. (Veterinarni Medicina, 2016, 61:475-496) and an automated chemistry analyzer available from Idexx is also capable of measuring serum globulin levels.

Suitably, increased serum globulin levels may be associated with a negative effect on reducing mortality risk. Accordingly, increased serum globulin levels may be associated an increased mortality risk.

### Serum Calcium

Serum Calcium is a measure of the total concentration of calcium in the blood. Calcium in the blood can be ionized, complexed, or protein-bound. Calcium is required in the body for a wide range of intracellular and extracellular functions, including muscular contractions and blood clotting, and is a major component of bone. Calcium levels that are too high can be as a result of certain cancers or bone disorders, and calcium levels that are too low can be as a result of kidney disease, pancreatitis or decreased serum albumin.

Methods of determining serum calcium levels, typically expressed in milligrams per decilitre (mg/dL), are well known in the art. F. Gran describes a colorimetric method for the determination of calcium in blood serum (Acta Physiologica Scandinavica; 1960, 49:192-197) and modern automated chemistry analyzers are capable of measurement of calcium levels in serum.

Suitably, increased serum calcium levels may be associated with a positive effect on reducing mortality risk. Accordingly, increased serum calcium levels may be associated a reduced mortality risk.

### Platelet Count

Platelets, also known as thrombocytes, are small cells that are components of the blood. They are small cells that lack a nucleus, and are produced from the cytoplasm of bone marrow cells known as megakaryocytes. Platelets help the clotting process to stop bleeding at the sites of damaged blood vessels. Platelet count levels that are above or below the normal range can indicate disorders or diseases. In particular, decreased platelet count can occur as a result of certain infections, cancer, immune system disorders or pancreatitis.

Methods of measuring platelet count, typically expressed in thousands of cells per microliter (10\hat{ }3/uL), are well known in the art. Platelet count can be done manually on a blood smear using staining and microscopy methods, but are commonly carried out as part of an automated complete blood count (CBC).

Suitably, increased platelet count may be associated with a negative effect on reducing mortality risk. Accordingly, increased platelet count may be associated an increased mortality risk.

### Red Blood Cell Count

Red blood cells, also known as red blood corpuscles, are the most abundant cells present in the blood. These cells do not contain a nucleus, and instead consist mainly of hemoglobin contained within the cell membrane to maximise their oxygen-carrying potential. Red blood cell counts that are above or below normal levels are indicative of disorders or disease. Low red blood count can indicate hemolysis, blood loss, or reduced production of red blood cells that can result from multiple causes. High red blood cell count can indicate a relative increase of red blood cells per volume of blood compared to normal, due to dehydration or increased red blood cell production.

Methods of measuring red blood cell count, typically expressed in thousands of cells per microliter (10\hat{ }3/uL), are well known in the art. Red blood cell count measurements can be done manually on a blood smear using microscopy, but are commonly carried out as part of an automated complete blood count (CBC).

Suitably, increased red blood cell count may be associated with a positive effect on reducing mortality risk. Accordingly, increased red blood cell count may be associated a reduced mortality risk.

### Combinations of Biomarkers

Whilst individual biomarkers may have predictive value in the methods of the present invention, the quality and/or the predictive power of the methods may be improved by combining values from multiple biomarkers.

Thus the present method may involve determining the level of at least two biomarkers from those described herein. For instance, the method may comprise determining the level of two or more biomarkers selected from white blood cell count, serum albumin, serum alkaline phosphatase, serum creatine kinase, haemoglobin, haematocrit, mean corpuscular haemoglobin, serum glucose, mean red cell volume, serum globulin, serum calcium, platelet count, and/or red blood cell count in one or more samples.

The term “one or more biomarkers” as used herein may include at least one, at least two, at least three, at least four, at least five, at least six, at least seven, at least eight, at least nine, at least ten, at least eleven, at least twelve or at least thirteen biomarkers.

The term “one or more biomarkers” as used herein may include one, two, three, four, five, six, seven, eight, nine, ten, eleven, twelve or thirteen biomarkers.

Suitably, the present method may comprise determining the level of white blood cell count, serum albumin and serum alkaline phosphatase in one or more samples. Advantageously, this combination of three biomarkers has been determined to provide a notable prediction of mortality risk and/or probability of a healthy lifespan. The predictive ability may be further increased by incorporating one or more of the additional biomarkers selected from serum creatine kinase, haemoglobin, haematocrit, mean corpuscular haemoglobin, serum glucose, mean red cell volume, serum globulin, serum calcium, platelet count, and red blood cell count.

Suitably, the present method may comprising determining the level of each of white blood cell count, serum albumin, serum alkaline phosphatase, serum creatine kinase, haemoglobin, haematocrit, mean corpuscular haemoglobin, serum glucose, mean red cell volume, serum globulin, serum calcium, platelet count, and red blood cell count in one or more samples.

### Comparison to a Reference or Control

The present method may further comprise a step of comparing the level of the individual biomarker(s) in the test sample to one or more reference or control values. The reference value may be associated with a pre-defined mortality risk and/or probability of a healthy lifespan. In some embodiments, the reference value is a value obtained previously for a subject or group of subjects with a known mortality outcome. The reference value may be based on an average level, e.g. a mean or median level, from a group of subjects with known chronological age, breed, sex and/or mortality outcome. Preferably, the reference value may be based on an average level, e.g. a mean or median level, from a group of subjects with known chronological age, breed, sex and mortality outcome.

Combining the Biomarker Levels with Further Measures and/or Characteristics

Suitably, the present method further comprises combining the level of the one or more biomarkers with one or more of the chronological age, breed and/or sex of the dog. By combining this information, an improved model is provided for the mortality risk and/or probability of a healthy lifespan of the dog.

In a preferred embodiment, levels of one or biomarkers as defined herein are determined for a sample from the dog and these levels are combined with the chronological age, breed and sex of the dog in order to determine a mortality risk and/or probability of a healthy lifespan for the dog.

Preferably, the mortality risk and/or probability of a healthy lifespan is represented as a phenotypic age (Phenoage), which is given by the following formula:

$$Phenoage = ln ( \frac{{γ}_{breed} * {e}^{xb} * \{ {e}^{γ * age} - 1 \}}{{e}^{\{ breed * {β}_{breed 2} \} + \{ sex * {β}_{sex 2} \} + {β}_{02}} * γ} + 1 ) * \frac{1}{{γ}_{breed}}$$

where xb is the sum of the value of each biomarker(s), sex and breed multiplied by their respective coefficients according to formula (2):

$$xb = {\sum }_{u = 1}^{p} {x}_{u} {β}_{u} + {β}_{0}$$

wherein sex is coded as a numerical value with 0 for female and 1 for male,
and wherein breed is coded as a numerical value with 0 for small breeds and 1 for medium breeds.

The coefficient value for each parameter typically depends on the measurement units of all the variables in the model. As would be understood by the skilled person, the exact value for each coefficient value will therefore depend on, for example, the number and nature of the different parameters used in the model and the nature of the training data provided. Accordingly, routine statistical methods may be applied to a training data set in order to arrive at coefficient values for use in above formula. Such methods include, for example, computation of two gompertz functions on a training set (e.g. where the status of the dog (alive or dead) is known), one that models survival as a function of the selected biomarkers, chronological age, breed class (small or medium dog) and sex (model 1) and a second function that only considers chronological age, breed class and sex (model 2). These models may be fit using the flexsurv package (v 2.1) in the R software environment.

Suitably, a negative coefficient for a given biomarker means that a higher level of the biomarker has a positive effect on reducing mortality risk and/or a lower level of the biomarker has a negative effect on reducing mortality risk. Suitably, a positive coefficient for a given biomarker means that a higher level of the biomarker has a negative effect on reducing mortality risk and/or a lower level of the biomarker has a positive effect on reducing mortality risk.

The phenotypic age may be defined as the time variable (“chronological age”) at which the survival probability of the animal given by model 2 is equal to the survival probability at their chronological age given by the model 1.

The phenotypic age (i.e. phenoage) of the dog may be expressed in terms of years, months, days, etc.

Preferably, the mortality risk and/or probability of a healthy lifespan is represented as the difference between phenoage and chronological age of the dog. This difference may be referred to as the phenoage advance of the dog.

For example, an increase in phenoage compared to chronological age may be indicative of an increased mortality risk for the dog. For example, a decrease in phenoage compared to chronological age may be indicative of a decreased mortality risk for the dog. As an illustration, the present inventors determined that the difference between phenoage and chronological age (phenoage advance) was associated with a significant increase in mortality risk, and the magnitude of the effect was calculated to be a hazard ratio of 1.75 for a 1 year increase in phenoage compared to chronological age (see Example 3). In other words, the inventors determined that a 1 year increase in phenoage vs chronological age was associated with a risk of mortality 75% higher at any given point in life.

### Subject Stratification

The mortality risk and/or probability of a healthy lifespan determined by the method of the present invention may also be compared to one or more pre-determined thresholds. Using such thresholds, subjects may be stratified into categories which are indicative of determined mortality risk and/or probability of a healthy lifespan, e.g. low, medium or high determined mortality risk. The extent of the divergence from the thresholds is useful to determine which subjects would benefit most from certain interventions. In this way, dietary intervention and modification of lifestyle can be optimised. The determined mortality risk and/or probability of a healthy lifespan may be presented in terms of a numerical score or percentage, which—for example—may be indicative of determined mortality risk and/or probability of a healthy lifespan compared to a control or reference population.

### Method for Selecting/Monitoring a Lifestyle or Dietary Regime of a Subject

In a further aspect, the present invention provides a method for selecting a lifestyle or dietary regime for a subject. The modification in lifestyle may be any change as described herein, e.g. a dietary intervention and/or a change in exercise regime. The modification in lifestyle may be administration of a therapeutic modality.

The lifestyle or dietary regime may be applied to the dog for any suitable period of time. After said period of time, the dog's mortality risk and/or probability of a healthy lifespan may be determined again using the present method in order to determine the efficacy of the lifestyle or dietary regime for reducing the mortality risk and/or increasing the probability of a healthy lifespan of the dog. By way of example, the lifestyle or dietary regime may be applied for at least 2, at least 4, at least 8, at least 16, at least 32, or at least 64 weeks. The lifestyle or dietary regime may be applied for at least 3, at least 6, at least 12, at least 24, at least 36, at least 48 or at least 60 months.

Preferably the modification is a dietary intervention as described herein. By the term “dietary intervention” it is meant an external factor applied to a subject which causes a change in the subject's diet. More preferably the dietary intervention includes the administration of at least dietary product or dietary regimen or a nutritional supplement.

The dietary intervention may be a meal, a regime of meals, a supplement or a regime of supplements.

The dietary intervention or dietary product described herein may be any suitable dietary regime, for example, a calorie-restricted diet, a senior diet, a low protein diet, a phosphorous diet, low protein diet, potassium supplement diet, polyunsaturated fatty acids (PUFA) supplement diet, anti-oxidant supplement diet, a vitamin B supplement diet, liquid diet, selenium supplement diet, omega 3-6 ratio diet, or diets supplemented with carnitine, branched chain amino acids or derivatives, nucleotides, nicotinamide precursors such as nicotinamide mononucleotide (MNM) or nicotinamide riboside (NR) or any combination of the above.

Suitably, the dietary intervention or dietary product may be a calorie-restricted diet, a senior diet, or a low protein diet. Suitably, the dietary intervention or dietary product may be a calorie-restricted diet. Suitably, the dietary intervention or dietary product may be a low protein diet.

A dietary intervention may be determined based on the baseline maintenance energy requirement (MER) of the dog. Suitably, the MER may be the amount of food that stabilizes the dog's body weight (less than 5% change over three weeks).

By way of example, it is generally understood that younger, growing dogs benefit from a high energy/high protein diet; however, older dogs may have a lower energy requirement and therefore diets can be appropriately modified. In particular, many manufacturers produce a ‘senior’ range of dog food which is lower in calories, higher in fibre but has suitable levels of protein and fat for an older dog.

Suitably, a calorie-restricted diet may comprise about 60%, about 65%, about 75% or about 80% of the dog's MER. Suitably, a calorie-restricted diet may comprise about 60% or about 75% of the dog's MER.

Suitably, a low-protein diet may comprise less than 20% protein (% dry matter). For example, a low-protein diet may comprise less than 15% or less than 10% protein (% dry matter).

These diets are generally recommended based upon the chronological age of a dog. For example, it may be recommended that a dog is switched to a senior diet around 7 or 8 years old. However, in the context of the present invention, the determination of an increased mortality risk and/or reduced probability of a healthy lifespan for a dog compared to what would be expected given its chronological age may allow a determination to switch the dog to a senior diet at an earlier age. In contrast, a dog with a reduced mortality risk and/or increased probability of a healthy lifespan compared to its chronological age may be able to stay on a high energy/high protein diet for longer.

The dietary intervention may comprise a food, supplement and/or drink that comprises a nutrient and/or bioactive that mimics the benefits of caloric restriction (CR) without limiting daily caloric intake. For example, the food, supplement and/or drink may comprise a functional ingredient(s) having CR-like benefits. Suitably, the food, supplement and/or drink may comprise an autophagy inducer. Suitably, the food, supplement and/or drink may comprise fruit and/or nuts (or extracts thereof). Suitable examples include, but are not limited to, pomegranate, strawberries, blackberries, camu-camu, walnuts, chestnuts, pistachios, pecans. Suitably, the food, supplement and/or drink may comprise probiotics with or without fruit extracts or nut extracts.

Modifying a lifestyle of the subject also includes indicating a need for the subject to change lifestyle, e.g. prescribing more exercise. Similar to a dietary intervention, the determination of an increased mortality risk and/or reduced probability of a healthy lifespan for a dog compared to what would be expected given its chronological age may allow a determination a switch the dog to an appropriate exercise regime.

Modifying a lifestyle of the subject also includes recommending a therapeutic modality or regimen. The therapeutic modality or regimen may be a modality useful in treating and/or preventing—for example—arthritis, dental diseases, endocrine disorders, heart disease, diabetes, liver disease, kidney disease, prostate disorders, cancer and behavioural or cognitive disorders. Suitably, prophylactic therapies may be administered to a dog identified as being at risk of such disorders due to increase mortality risk (phenoage) and/or on the basis of particular biomarkers which are known to be associated with disease-relevant pathways. In other embodiments, dogs determined to be at risk of certain conditions (due to increase mortality risk (phenoage) and/or on the basis of particular biomarkers which are known to be associated with disease-relevant pathways) may be monitored more regularly so that diagnosis and treatment can begin as early as possible.

The present invention may thus advantageously enable the identification of dogs that are expected to respond particularly well to a given intervention (e.g. lifestyle or dietary regime). The intervention can thus be applied in a more targeted manner to dogs that are expected to respond.

The invention further provides a method for determining the efficacy of a lifestyle or dietary regime for reducing the mortality risk and/or increasing the probability of a healthy lifespan for a dog, said method comprising:
a. determining a first mortality risk and/or probability of a healthy lifespan for the dog according to the method of the first aspect of the invention;
b. applying a lifestyle or dietary regime to the dog;
c. after a time period of applying the lifestyle or dietary regime to the dog, determining a second mortality risk and/or probability of a healthy lifespan for the dog according to the method of the first aspect of the invention;
d. determining if there has been a change in the first and second mortality risk and/or probability of a healthy lifespan for the dog after the time period of following the lifestyle or dietary regime.

The invention also provides a method for determining the efficacy of a lifestyle or dietary regime for reducing the mortality risk and/or increasing the probability of a healthy lifespan determined for a dog, said method comprising:
a. applying a lifestyle or dietary regime to the dog, wherein the lifestyle or dietary regime has been selected according to a method comprising performing the method according to the first aspect of the invention; and selecting a suitable lifestyle or dietary regime based on the mortality risk and/or probability of a healthy lifespan determined;
b. determining a second mortality risk and/or probability of a healthy lifespan for the dog by performing the method of the first aspect of the invention after a time period of applying the lifestyle or dietary regime to the dog;
c. determining if there has been a change in mortality risk and/or probability of a healthy lifespan for the dog after the time period of following the lifestyle or dietary regime.

The invention further provides a method for determining the efficacy of a lifestyle or dietary regime for reducing the mortality risk and/or increasing the probability of a healthy lifespan of a dog, said method comprising:
a. selecting a lifestyle or dietary regime for the dog according to a method comprising performing the method according to the first aspect of the invention; and selecting a suitable lifestyle or dietary regime based on the mortality risk and/or probability of a healthy lifespan determined;
b. applying the lifestyle or dietary regime to the dog;
c. after a time period of applying the lifestyle or dietary regime to the dog, determining a second mortality risk and/or probability of a healthy lifespan for the dog according to the method of the first aspect of the invention;
d. determining if there has been a change in mortality risk for the dog between step a. and step c.

A reduction in the second (or subsequent) mortality risk determined for the dog compared to the first (or earlier) mortality risk determined for the dog after a period of applying the lifestyle or dietary regime is indicative that the lifestyle or dietary regime is effective in reducing the mortality risk for the dog.

The mortality risk and/or probability of a healthy lifespan for the dog may be determined prior to and after the lifestyle or dietary regime has been applied to the dog. The mortality risk and/or probability of a healthy lifespan for the dog may also be determined at predetermined times throughout the application of the lifestyle or dietary regime. These predetermined times may be periodic throughout the lifestyle or dietary regime, e.g. every day or three days, every week, every two weeks, every month, every two months, every 6 months, every year or every two years. The predetermined times may depend on the subject being tested. Suitably, the lifestyle or dietary regime may have been applied to the dog for a period before the first mortality risk and/or probability of a healthy lifespan is determined; however, the effectiveness of the lifestyle or dietary regime for reducing mortality risk and/or increasing the probability of a healthy lifespan may still be monitored by determining a mortality risk and/or probability of a healthy lifespan at two or more predetermined times during the application of the lifestyle or dietary regime.

### Use of a Dietary Intervention

In one aspect, the present invention provides a dietary intervention for use in reducing the mortality risk of a dog and/or increasing the probability of a healthy lifespan, wherein the dietary intervention is administered to a dog with a mortality risk and/or probability of a healthy lifespan determined by the present method.

In another aspect, the present invention provides the use of a dietary intervention to reduce the predicted mortality risk of a dog and/or increasing the probability of a healthy lifespan, wherein the dietary intervention is administered to a dog with a mortality and/or probability of a healthy lifespan risk determined by the present method.

As described herein, the dietary intervention may be a dietary product or dietary regimen or a nutritional supplement.

### Computer Program Product

The present methods may be performed using a computer. Accordingly, the present methods may be performed in silico.

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to determine the mortality risk and/or probability of a healthy lifespan of a dog as described herein.

In another aspect, the present invention provides a computer program product comprising computer implementable instructions for causing a device to determine the mortality risk and/or probability of a healthy lifespan of a dig given the levels of one or more biomarkers from the user, wherein the biomarkers are selected from the one or more biomarkers as defined herein. Preferably, the biomarker levels are fasting levels. The computer program product may also be given additional parameters or characteristics for the dog. As described herein, the additional parameters or characteristics may include chronological age, breed and sex.

In one embodiment, the user inputs into the device levels of one or more of the biomarkers as defined herein, optionally along with chronological age, breed and sex. The device then processes this information and provides a determination of a mortality risk and/or probability of a healthy lifespan for the dog.

The device may generally be a server on a network. However, any device may be used as long as it can process biomarker data and/or additional parameters or characteristic data using a processor, a central processing unit (CPU) or the like. The device may, for example, be a smartphone, a tablet terminal or a personal computer and output information indicating the determined mortality risk and/or probability of a healthy lifespan for the dog. The determined mortality risk and/or probability of a healthy lifespan may be presented in terms of a numerical score or percentage, which—for example—may be indicative of determined mortality risk and/or probability of a healthy lifespan compared to a control or reference population.

Those skilled in the art will understand that they can freely combine all features of the present invention described herein, without departing from the scope of the invention as disclosed.

### EXAMPLES

The invention will now be further described by way of examples, which are meant to serve to assist the skilled person in carrying out the invention and are not intended in any way to limit the scope of the invention.

### Example 1—Determination of Blood Biomarkers Associated with Mortality Risk in Dogs

Predictive blood biomarkers were determined from a biomarker panel consisting of a standard clinical complete blood count (cbc) and standard clinical blood chemistry analysis. Serum samples were taken after overnight fasting and measured using standard veterinary clinical practice.

TABLE 1
Clinical complete blood count (cbc)
and clinical blood chemistry analysis

| Parameter name | Unit of measure |
|---|---|
| Hematocrit | % |
| Hemoglobin | g/dL |
| Mean Corpuscular Hemoglobin | pg |
| Mean Corpuscular Hemoglobin concentration | g/dL |
| Mean Red Cell Volume | fL |
| Platelet | 10\hat{ }3/uL |
| Red blood cells | 10\hat{ }3/uL |
| White blood cells | 10\hat{ }3/uL |
| Serum Albumin Plus | g/dL |
| Serum Alkaline Phosphatase * | U/L |
| Serum ALT * | U/L |
| Serum AST * | U/L |
| Serum Calcium | mg/dL |
| Serum Chloride | mmol/L |
| Serum Cholesterol | mg/dL |
| Serum Cretaine Kinase * | IU/L |
| Serum Creatinine, Jaffe Method * | mg/dL |
| Serum GGT * | g/dL |
| Serum Globulin | g/dL |
| Serum Glucose | mg/dL |
| Serum Magnesium | mg/dL |
| Serum Phosphorus | mg/dL |
| Serum Potassium | mmol/L |
| Serum Sodium | mmol/L |
| Serum Total Bilirubin * | mg/dL |
| Serum Total Protein | g/dL |
| Serum Triglycerides * | mg/dL |
| Serum Urea Nitrogen * | mg/dL |

* value were log-transformed using natural logarithm.

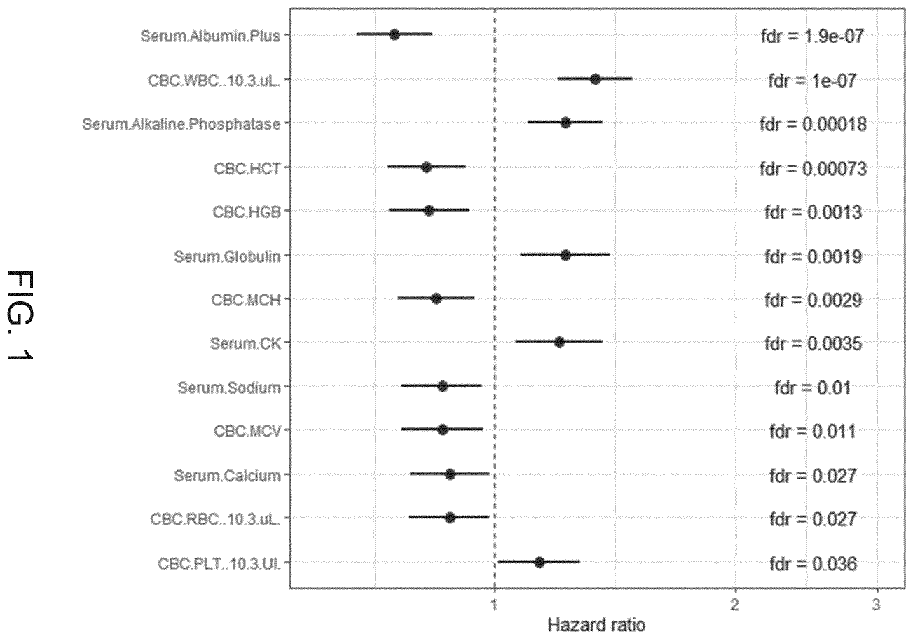

We used a longitudinal study of dogs for which we have repeated measurement of these parameters as well as information about the status of the dog (alive or dead), their sex and their breed. We first categorized breeds as small or medium based on the average weight of adult dogs of this breed (below 10 kg or above 10 kg, respectively). Then we organized the data using the R programming language. For each dog, we recorded the biomarkers as time dependent covariates using time intervals open on the left and closed on the right (i.e. (tstart, tstop]), where the biomarker information corresponds to the start of the interval and the event (alive or dead) is recorded as the last tstop value. For this purpose, we used the tmerge function of the survival package in R (v. 3.2-13). Then, we fit a cox proportional hazard model to this data individually for each of the 28 biomarkers, including sex and breed class (small or medium). We then adjusted the p. value of each parameter to account for multiple comparison (by false discovery rate (fdr)) and selected features with an adjusted fdr below 0.05 (FIG. 1).

Using this method, we identified 13 biomarkers that are individually predictive of the survival probability in dogs:
White blood cells count (10\hat{ }3 per ul)
Serum Albumin (g/dL)
Serum Alkaline phosphatase (U/L, In-transformed)
Serum creatine Kinase (IU/L, In-transformed)
Hemoglobin (g/dL)
Hematocrit (%)
Mean Corpuscular Hemoglobin (pg)
Serum Sodium (mmol/L)
Mean Red Cell Volume (fL)
Serum Globulin (g/dL)
Serum Calcium (mg/dL)
Serum Platelet Count (10\hat{ }3/uL)
Red Blood Cell Count (10\hat{ }3/uL)

### Example 2—Multi-Parameter Model for Predicting Mortality Risk

Next, we constructed the best model that would consider multiple parameters simultaneously, as this is more likely to cover a wide range of organ dysfunctions that occur with age. However, selecting several features that might be correlated with each other is subject to bias. To avoid this issue, we used a penalized regression method using the glmnet package (v4.1-3). We fit a LASSO-penalized cox proportional hazard model on data and used 20-fold cross validation to compare different values of the penalization parameter lambda. This approach leads to the selection of the top 10 most predictive blood biomarkers for survival, by order of importance as shown below:
White blood cells count (10\hat{ }3 per ul)
Serum Albumin (g/dL)
Serum Alkaline phosphatase (U/L, In-transformed)
Serum creatine Kinase (IU/L, In-transformed)
Hemoglobin (g/dL)
Hematocrit (%)
Mean Corpuscular Hemoglobin (pg)
Serum Glucose (mg/dL)
Mean Red Cell Volume (fL)
Serum Globulin (g/dL)

We also found that the first 3 biomarkers from this list are the most predictive and that the performance can be increased by incorporating each of the next 7 biomarkers.

To extract the phenotypic age of the animal, we computed two different gompertz functions on our training set, one that models survival as a function of the selected biomarkers, age, breed class (small or medium dog) and sex (model 1) and a second function that only considers age, breed class and sex (model 2). These models were fit using the flexsurv package (v 2.1). The phenotypic age was defined as the time variable (“age”) at which the survival probability of the animal given by model 2 is equal to the survival probability at their chronological age given by the model 1. This leads to a mathematical function connecting the blood biomarkers to the phenoage and is given by the following formula:

$$Phenoage = ln ( \frac{{γ}_{breed} * {e}^{xb} * \{ {e}^{γ * age} - 1 \}}{{e}^{\{ breed * {β}_{breed 2} \} + \{ sex * {β}_{sex 2} \} + {β}_{02}} * γ} + 1 ) * \frac{1}{{γ}_{breed}}$$

Where xb is the sum of the value of each biomarkers, sex and breed multiplied by their respective coefficients. Sex and breeds are coded as numerical value with 0 for female and 1 for males and 0 for small breeds and 1 for medium breeds. The coefficients are given by the two gompertz function trained on our training sets.

As an example, the coefficients, as well as the γ and γ_{breed} values have been measured from our training set for the complete list of biomarkers and are given in Table 2.

$$xb = {\sum }_{u = 1}^{p} {x}_{u} {β}_{u} + {β}_{0}$$

TABLE 2
Coefficients and γ and γ_{breed} values
have been measured from training set

| | Coefficient |
|---|---|
| γ | 0.491790219 |
| β | −6.036261473 |
| β White blood cells count | 0.091862564 |
| β Hemoglobin | −0.009131623 |
| β Mean Red Cell Volume | −0.007486146 |
| β Hematocrit | −0.018418391 |
| β Mean Corpuscular Hemoglobin | −0.128195615 |
| β Serum Glucose | 0.009169677 |
| β Serum Globulin | 0.132755858 |
| β Serum Creatine Kinase | 0.332818902 |
| β Serum Albumin | −0.744060565 |
| β Serum Alkaline Phosphatase | 0.262594338 |
| β breed | 1.138018960 |
| β Sex | 0.151826455 |
| γ_{breed} | 0.5668399 |
| β₀₂ | −9.5204440 |
| β_{breed2} | 1.2299804 |
| βₛₑₓ₂ | 0.2678798 |

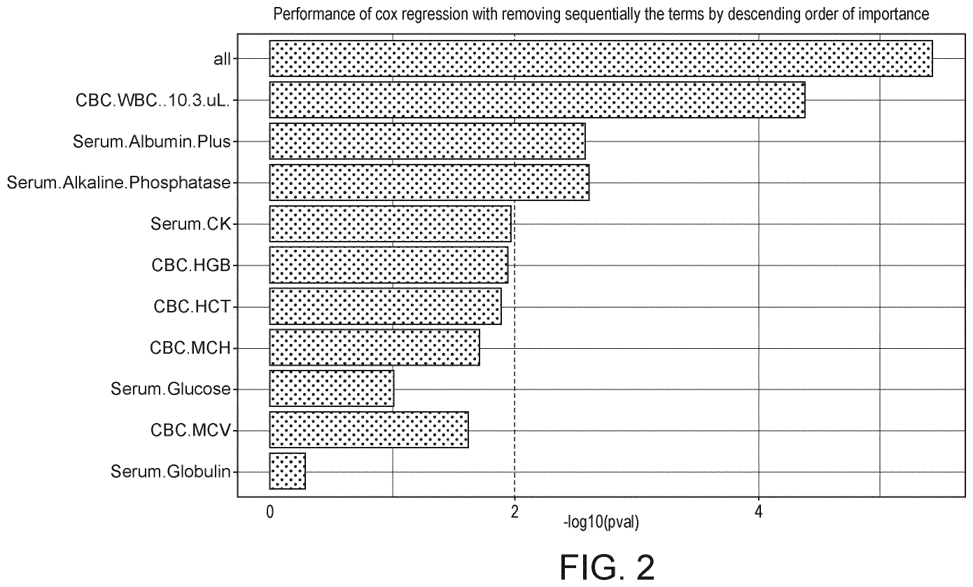

Further, by reducing the set of 10 biomarkers by systematically removing one biomarker, starting for the top of the list, we observed a reduction in the strength of the survival prediction (p value). The drop was most pronounced with the first parameters, confirming their biggest contribution, but we observed a change in quality of prediction by each reduction of the set, showing that each parameter contributes to the overall prediction (FIG. 2).

### Example 3—Application of Phenoage for Predicting Mortality Risk

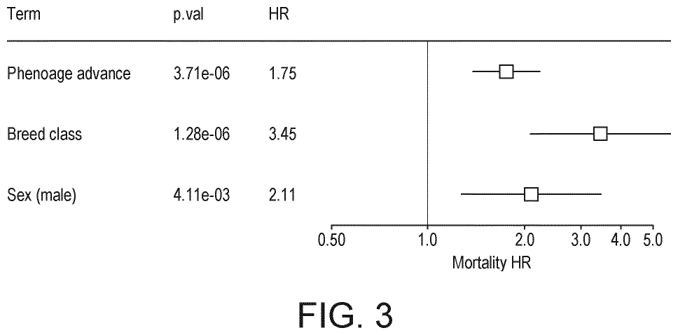

It was subsequently demonstrated that when applying the resulting phenoage to a test set consisting of only dogs that were not used during training of the algorithm, the difference between phenoage and chronological age (phenoage advance) was associated with a significant increase in mortality risk. The magnitude of the effect was calculated to be a hazard ratio of 1.75 for a 1 year increase in phenoage compared to chronological age (FIG. 3).

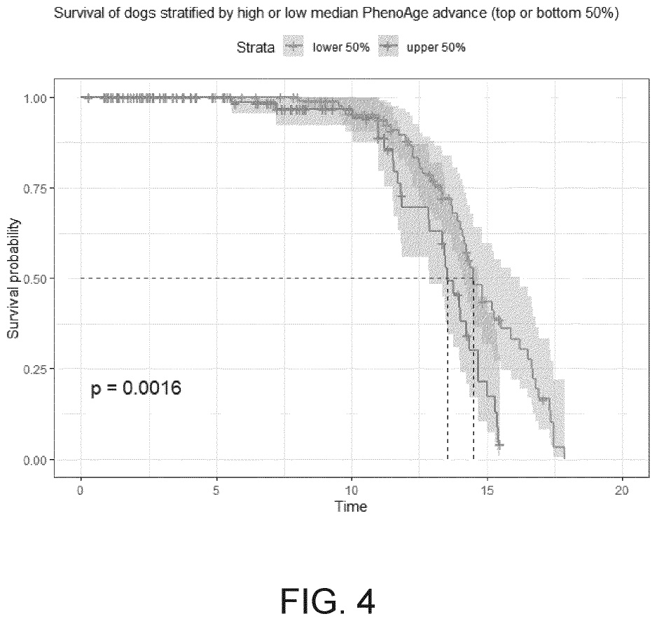

In addition, survival of dogs stratified by high or low median PhenoAge advance showed a statistically significant survival differences between the top 50% vs bottom 50% (FIG. 4).

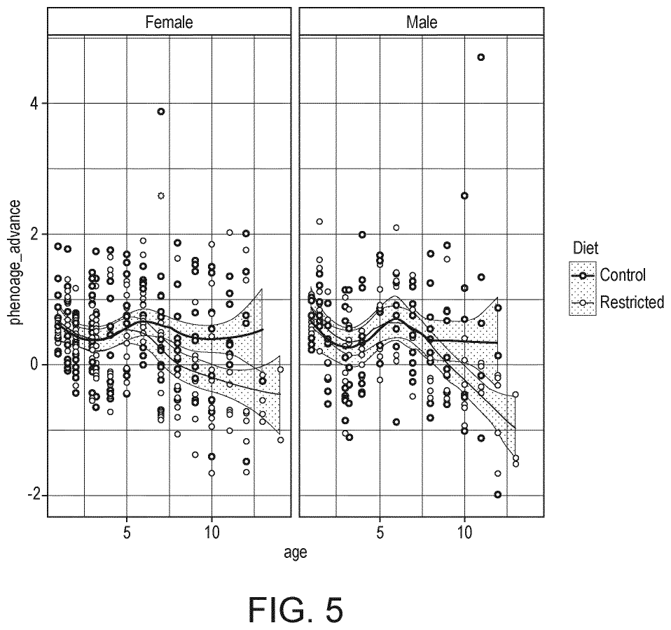

Phenoage advance (delta with chronological age) changes mid-life with a calorie-restricted diet (75% baseline maintenance energy requirement (MER)). This changes earlier in females than males (FIG. 5).

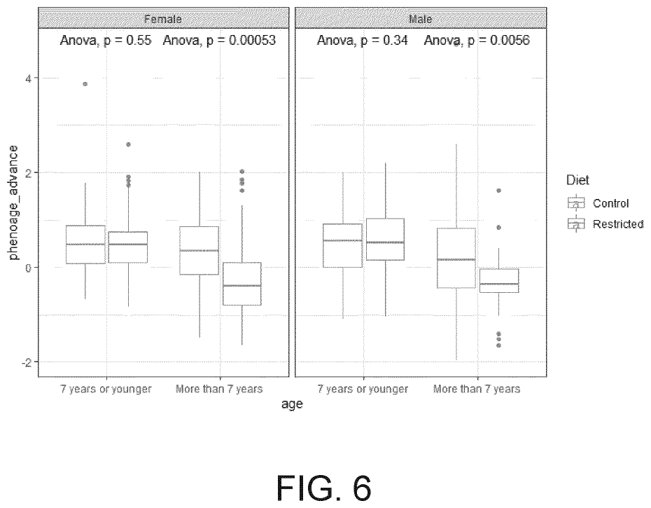

Stratification of under 7 and over 7 year old dogs reveals a significant difference in older compared to younger dogs. This is more severe in females than males (FIG. 6).

### Example 4—Reduced Protein Diet Reduces PhenoAge Advance

### Dogs

Thirty dogs with a body condition score (BCS) of 7 or higher were recruited in this weight loss study. Each dog's baseline maintenance energy requirement (MER) was determined as the amount of food that stabilized the dog's body weight (less than 5% change over three weeks). Then each dog's baseline % body fat was determined by DEXA. The dogs were randomized into 2 groups based on baseline body weight, MER, age, gender and % body fat with 15 dogs per group.

### Test Diets

The two diets had comparable metabolizable energy (ME), but differed in protein, carbohydrate, fat and fiber.

| | High protein low | Moderate protein moderate |
|---|---|---|
| | carbohydrate diet | carbohydrate diet |
| Moisture (% as fed) | 8.07 | 8.09 |
| Protein (% as fed) | 48.70 | 26.47 |
| CHO (% as fed) | 22.19 | 34.12 |
| Fat (% as fed) | 10.1 | 14.73 |
| Crude fiber (% as | 5.0 | 11.4 |
| fed) | | |
| ME (kcal/kg) | 3150 | 3181 |

### Feeding Instruction

Dogs in both groups were fed 75% of their baseline MERs during the first 4 months of the study and 60% of their baseline MERs during the last 2 months of the study.

### Blood Sample Collection and Analysis

Serum samples were collected at baseline, 2, 4 and 6 months of the study. Complete blood count (CBC) and blood chemistry panel analyses were performed on those serum samples at the end of the study.

### Results

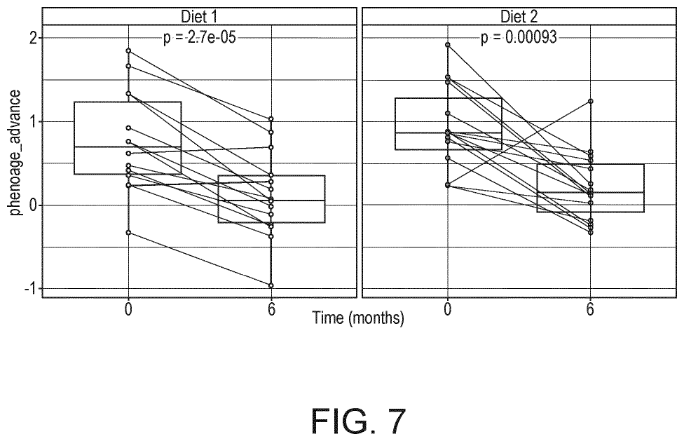

As shown in FIG. 7, compared to baseline, all but one dog showed a decrease in phenoage advance, defined as the difference between a dog's phenoage and their chronological age following a 6 month period of the weight loss diet. The average difference in phenoage advance between baseline and after intervention is 0.7 years and is significant in a paired t-test (p=0.00093). The ages of dogs in the study ranged from 3 to 11 years. We detected no correlation between the gain in PhenoAge advance and the age of the dog at the beginning of the study, showing that benefits extend across a wide range of dog life stages. Further, although the two diets had quite different protein levels, the effects on PhenoAge were comparable between the two diets, suggesting that reduction in caloric intake led to younger PhenoAge regardless of the macronutrient ratios in dogs.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for determining a mortality risk and/or probability of a healthy lifespan of a dog; said method comprising determining the level of one or more biomarker(s) in one or more samples obtained from the dog, wherein the one or more biomarker(s) is selected from the group consisting of white blood cell count, serum albumin, serum alkaline phosphatase, serum creatine kinase, haemoglobin, haematocrit, mean corpuscular haemoglobin, serum glucose, mean red cell volume, serum globulin, serum calcium, platelet count, and red blood cell count.

2. The method according to claim 1 wherein the biomarker is white blood cell count.

3. The method according to claim 1 wherein the biomarker is serum albumin.

4. The method according to claim 1 wherein the biomarker is serum alkaline phosphatase.

5. The method according to claim 1 wherein the method comprises measuring each of white blood cell count, serum albumin and serum alkaline phosphatase.

6. The method according to claim 1 wherein the method comprises measuring each of white blood cells count, serum albumin, serum alkaline phosphatase, serum creatine kinase, haemoglobin, haematocrit, mean corpuscular haemoglobin, serum glucose, mean red cell volume, and serum globulin.

7. The method according to claim 1 wherein the one or more sample(s) is a blood sample.

8. The method according to claim 1 wherein the method further comprises combining the level of the one or more biomarker(s) with one or more of the chronological age, breed and/or sex of the dog.

9. The method according to claim 1 wherein the dog is classified as small, medium, large or giant and/or as robust or athletic.

10. A method for determining a mortality risk and/or probability of a healthy lifespan of a dog; said method comprising:
a. determining the level of the following biomarkers; white blood cell count, serum albumin, serum alkaline phosphatase, serum creatine kinase, haemoglobin, haematocrit, mean corpuscular haemoglobin, serum glucose, mean red cell volume, and serum globulin in one or more samples obtained from the dog; and
b. determining a phenotypic age (Phenoage) of the dog using formula (1):
$$Phenoage = ln ( \frac{{γ}_{breed} * {e}^{xb} * \{ {e}^{γ * age} - 1 \}}{{e}^{\{ breed * {β}_{breed 2} \} + \{ sex * {β}_{sex 2} \} + {β}_{02}} * γ} + 1 ) * \frac{1}{{γ}_{breed}}$$
where xb is the sum of the value of each biomarker(s), sex and breed multiplied by their respective coefficients according to formula (2):
$$xb = {\sum }_{u = 1}^{p} {x}_{u} {β}_{u} + {β}_{0}$$
wherein sex is coded as a numerical value with 0 for female and 1 for males,
wherein breed is coded as a numerical value with 0 for small breeds and 1 for medium breeds; and
c. using the phenotypic age to determine a mortality risk and/or probability of a healthy lifespan for the dog.

11. The method according to claim 10 wherein determining that the phenoage of the dog is greater than its chronological age is indicative of a higher mortality risk and/or a reduced probability of a healthy lifespan.

12. The method according to claim 10 wherein determining that the phenoage of the dog is less than its chronological age is indicative of a lower mortality risk and/or an increased probability of a healthy lifespan.

13. The method according to claim 1 wherein the method is performed on one or more samples obtained before and after a time interval and determining if there has been a change in the mortality risk and/or probability of a healthy lifespan of the dog during the time interval.

14. (canceled)

15. A method for selecting a lifestyle or dietary regime for a dog, the method comprising:
a. performing the method for determining a mortality risk and/or probability of a healthy lifespan of a dog; said method comprising determining the level of one or more biomarker(s) in one or more samples obtained from the dog, wherein the one or more biomarker(s) is selected from the group consisting of white blood cell count, serum albumin, serum alkaline phosphatase, serum creatine kinase, haemoglobin, haematocrit, mean corpuscular haemoglobin, serum glucose, mean red cell volume, serum globulin, serum calcium, platelet count, and red blood cell count; and
b. selecting a suitable lifestyle or dietary regime based on the mortality risk and/or probability of a healthy lifespan determined in step a.

16. A method according to claim 15 wherein the lifestyle or dietary regime is a dietary intervention.

17. A method according to claim 15 wherein the dietary intervention is a calorie-restricted diet, a senior diet or a low protein diet.

18-41. (canceled)
